(19) United States
(12) Patent Application Publication
(10) Pub. No.: US 2024/0374276 A1
(43) Pub. Date: Nov. 14, 2024

(54) MEDICAL SYSTEM FOR REVISION SINUS SURGERY

(71) Applicant: Dalent, LLC, Miami, FL (US)

(72) Inventors: Agustin Arrieta, Pinecrest, FL (US); Peter Flores, Miami, FL (US)

(21) Appl. No.: 18/657,473

(22) Filed: May 7, 2024

Related U.S. Application Data
(60) Provisional application No. 63/508,101, filed on Jun. 14, 2023.
(60) Provisional application No. 63/465,212, filed on May 9, 2023.

(51) Int. Cl. A61B 17/24; A61M 29/02

(52) U.S. Cl. CPC A61B 17/24; A61M 29/02; A61M 2210/0681

(57) ABSTRACT

Systems and methods provided for comprehensive eustachian tube and/or sinus treatment that integrate a dilation balloon, a dilation sleeve and tip, an image navigation system, a sinus stent, an intranasal scanner, and/or an irrigation system into a single device. Embodiments offer versatile and efficient solutions for diagnosing, treating, and monitoring eustachian tube and/or sinus conditions by providing precise dilation, accurate image-guided navigation, optimal stent placement, real-time intranasal scanning, and/or effective irrigation capabilities. This innovative approach significantly enhances the quality of patient care, simplifies the surgical process, and improves overall treatment outcomes in the field of eustachian tube and/or sinus treatments.

### CROSS-REFERENCE TO RELATED APPLICATIONS

This application claims priority to U.S. Provisional Patent Application No. 63/508,101, filed Jun. 14, 2023, and U.S. Provisional Patent Application No. 63/465,212, filed May 9, 2023, each of which are incorporated herein by reference in their entireties. Any and all applications, if any, for which a foreign or domestic priority claim is identified in the Application Data Sheet of the present application are hereby incorporated by reference under 37 CFR 1.57.

This application has subject matter related to U.S. Pat. No. 10,322,269, issued Jun. 28, 2019, U.S. patent application Ser. No. 14/599,817, filed Jan. 19, 2015, and U.S. patent application Ser. No. 17/615,758, filed Dec. 1, 2021, each of which are incorporated by reference herein in their entireties.

### BACKGROUND

### Field

Embodiments of the invention are directed to a medical system that provides treatment solutions to surgeons and patients for revision sinus surgery and/or eustachian tube therapy, comprising a balloon dilation device, an endoscope and image navigation system, a dilation sleeve, an intranasal scanner, an absorbable stent, a suction device, and/or an irrigator.

### Description of the Related Art

Paranasal sinuses of the human body comprise a plurality of right and left sinus cavities including frontal, ethmoid, sphenoid and maxillary sinus cavities. The maxillary sinuses are the largest and most common site for sinus infection. In addition, the paranasal sinuses are lined with mucus-producing tissue that is in communicating relation with the nasal cavity. Mucus produced by the indicated tissue slowly drains out of each sinus through an opening or ostium. If the mucus draining tissue of a corresponding passageway becomes inflamed, the corresponding cavities through the passageways can become blocked. Such blockage interferes with the drainage of mucous typically resulting in occlusion of the sinus ostium and mucosal congestion within the paranasal sinuses. Chronic congestion, of this type, within the sinuses can cause damage to the tissue that lines the sinus and a resulting sinus infection.

### SUMMARY

Embodiments of the invention are directed towards the production of innovative and proprietary diagnostic, intraoperative, and in-office devices for the treatment of eustachian tubes and/or recalcitrant sinusitis, with a particular focus on the anatomically complex and surgically challenging frontal sinus. The frontal sinus outflow tract's anatomy contributes to the narrow and limited area for surgical dissection, thereby increasing the risk of major complications and stenosis. In various embodiments, reference to a sinus herein includes and contemplates reference to an eustachian tube. Furthermore, postoperative observation and wound care are difficult for both surgeons and patients to access, as evidenced by a 15-20% reported postoperative revision rate for the frontal sinus. Upon diagnosing a problematic postoperative sinus, the invention provides surgeons with multiple instrument options for addressing the issue. An initial choice of treatment includes a balloon sinus dilation (BSD) device, which has become a popular surgical therapy for frontal sinus revision due to its reduced risk of complications, adaptability to in-office or operating room settings, and favorable reimbursement. The inventive BSD solution is uniquely qualified for revision sinus surgery (including the eustachian tube), as it offers dissection, suction, and anatomic confirmation within a single device.

In one embodiment, an integrated sinus dilation and stent placement device is provided for use in sinus surgery. In one embodiment, an integrated sinus dilation and stent placement device is provided for use in an eustachian tube. The device comprises a lead dilation balloon and an inferiorly placed balloon for repeat dilation and stent placement. The device further includes an hourglass-shaped inferior balloon, which facilitates the placement of a stent in the most advantageous position to maintain patency in the frontal sinus drainage pathway.

In one embodiment, an endoscopic and navigation-enabled device for use in sinus/eustachian tube surgery is provided which combines an endoscope and image guidance navigation in a single handheld instrument. The device can be coupled with a dilation balloon sheath or incorporate a dilation balloon directly into the body of the device. The device features a 130-degree angle handle designed for optimal ergonomics and mechanical advantage during surgery. The navigation technology is located at the distal tip of the device and may be implemented via an electrode/chip or wire.

In one embodiment, a sinus/eustachian tube dilation sleeve is provided that is equipped with image guidance navigation through the body of the sleeve leading to the tip of the sinus/eustachian tube dilation sleeve. This is accomplished via the placement of a wire or electrode in the sleeve and/or tip, allowing for real-time tracking and guidance during the procedure. The sinus/eustachian tube dilation sleeve further includes the capability of the tip to hold an illuminating element, which may be a wire or fluorescent dye. This feature provides an additional signal to confirm accurate anatomic placement of the device and minimizes the risk of complications during the procedure. In addition, the present invention features a navigation sleeve that can be placed over multiple different surgical instruments. This design confers image guidance capabilities to previously non-navigation instruments, increasing the versatility and adaptability of the device in various surgical settings.

For example, a device we will refer to as a Navendosleeve device is an innovative amalgamation of advanced imaging, navigation, and sinus sleeve technology, offers manifold benefits to both surgeons and patients. This novel system facilitates enhanced and direct visualization of the targeted sinus ostia/eustachian tube and post-treatment inspection of the dilated sinus/eustachian tube cavity, acting as a virtual second hand to the surgeon. By significantly enhancing surgical efficiency and minimizing tissue trauma, it contributes to improved patient outcomes. Furthermore, as a key component of a fully disposable surgical kit, the Navendosleeve streamlines instrumentation handling and accelerates turnover times between procedures. Uniquely, it is the only balloon sinus dilation (BSD) system enabling surgeons to confirm anatomical position using a tri-modal approach, combining direct visualization, light confirmation, and navigation. This provides an unprecedented level of precision and assurance during surgical interventions.

In one embodiment, an intranasal/sinus scanner is provided for use intraoperatively to identify residual sinus disease or obstruction and postoperatively in the office for surveillance and follow-up. The device functions similarly to current dental intraoral scanners, capturing direct optical impressions and creating a 3D surface model of the sinus anatomy. The scanner generates a view of the sinus ostia from an intranasal viewpoint, allowing for accurate assessment and early detection of treatment needs.

In one embodiment, an adjunct to sinus dilation in revision sinus surgery is stent placement in the sinus opening. For these patients, the device provides two additional treatment options, including a dedicated BSD device and stent for precise dilation and placement. The need for a simple, effective, and lower-cost stenting solution has been identified through relationships with sinus surgeons and an in-depth understanding of the reimbursement landscape, enabling the treatment of problematic postoperative sinuses in an office setting.

In one embodiment, postoperative care treatment for initial and revision sinus surgery patients involves irrigation and debridement of the surgical site. This embodiment relates to a suction irrigation device designed for the nasal and sinus cavities, including the eustachian tube, comprising an irrigant chamber, a propulsion system, and a malleable distal tip. The malleable distal tip allows surgeons to manipulate and bend the tip as needed for targeting specific sinus regions. The device also features detachable distal tips with various fixed angles to accommodate diverse surgical requirements and patient anatomies.

In various embodiments, the systems and methods disclosed apply to treatments related to otology. For example, the Navendosleeve, a low-profile and malleable imaging device, is an important instrument in the rapidly evolving field of otology, which is increasingly adopting endoscopic technology for minimally invasive ear surgery. By enabling otologic surgeons to utilize the external auditory canal as a minimal access surgical corridor, the Navendosleeve provides a wide-angle, superiorly illuminated view of the surgical field, facilitating visualization around corners and into hidden recesses. Its optimal size, weight, and flexibility not only promote ear surgery but also significantly mitigate hand fatigue, a common issue with standard endoscope systems. The introduction of a disposable variant of the Navendosleeve further enhances its utility, enabling a seamless transition of otologic surgeries into office settings and reducing the need for endoscope angle exchanges. It has demonstrated its significant benefits across a range of otologic procedures, such as Eustachian tube dilation, middle ear exploration, tube placement, myringoplasty, tympanoplasty, cholesteatoma treatment, middle ear neoplasm treatment, ossicular chain reconstruction, and schwannoma resection. The Navendosleeve, with its advanced features, offers maxillofacial surgeons improved access to the operative field, thus enhancing surgical precision and efficiency. Its superior visualization capabilities, coupled with the provision of multiple perspectives of the surgical field, significantly enhance the precision of procedures such as fracture repair, orthognathic osteotomy placement and fixation, and temporomandibular joint (TMJ) exploration and intervention. This revolutionary tool also contributes to surgical efficiency by being part of a disposable imaging and instrumentation system, potentially increasing the transition of cases into office settings, with TMJ joint exploration and intervention being a possible initial point of entry. Furthermore, the Navendosleeve's smaller profile, reduced weight, and malleability lead to increased anatomical precision, facilitating confirmation of tissue reduction and plate placement. The integration of navigation technology would further enhance these precision-driven capabilities.

In some variants, a sinus surgical device for providing a surgeon the ability to perform simultaneous or sequential dilation of an obstructed sinus and placement of one or more stents for a patient is disclosed herein. The device can include a handle. The device can include a shaft extending from a distal end of the handle. The distal end of the shaft can be inserted into a sinus cavity of the patient. The distal end of the shaft can include a lead dilation balloon. The distal end can include a stent placement mechanism to facilitate the positioning and deployment of one or more stents. The device can be designed for manual operation, enabling a surgeon to perform both dilation and stent placement in a simultaneous or sequential maneuver.

In some variants, the lead dilation balloon is a distal balloon and an inferiorly placed balloon is a proximal balloon that can be used for repeated dilation and stent placement.

In some variants, the proximal balloon can include an hourglass shape that can facilitate placement of the one or more stents in a desired position to maintain patency in a frontal sinus drainage pathway.

In some variants, the device can include two separate inflation ports that can enable the surgeon to individually inflate the lead dilation balloon and the inferiorly placed balloon.

In some variants, the distal end of the shaft can include a navigation wire, tip, chip, or electrode.

In some variants, the distal end of the shaft can hold an illuminating element, which can be provided by a wire or fluorescent dye.

In some variants, an endoscopic and navigation-enabled device for sinus surgery is disclosed herein. The device can include a handle. The device can include a shaft extending from a distal end of the handle. The device can include a distal tip of the shaft that incorporates an endoscope and image guidance navigation technology. The device can be used with or without a dilation balloon, allowing for an integrated and efficient surgical instrument for the surgeon.

In some variants, the shaft can have a 130-150 (e.g., 130, 140, 145, 150) degree angle with respect to the handle to place the surgeon's hands in a desired position.

In some variants, the body of the shaft can be hollow so that other medical instruments may be coupled with the hollow shaft.

In some variants, the hollow shaft can be designed to be capable of being coupled with a dilation balloon sheath.

In some variants, the body of the shaft can be made of malleable materials so that the shaft is bendable to target desired locations.

In some variants, the distal tip of the shaft can hold an illuminating element.

In some variants, the navigation technology can be provided by the placement of an electrode, chip, or wire at the distal tip of the shaft.

In some variants, a device for facilitating sinus surgery is disclosed herein. The device can include a sleeve defining an elongated structure with an interior cavity that can receive various surgical instruments. The device can include an image guidance navigation system integrated within the sleeve body and extending along the length of the sleeve, terminating at the distal end of the sleeve. The sleeve can be made of malleable materials so that the sleeve is adaptable for use with other surgical instruments. The image guidance navigation system can include wires and/or electrodes, situated within the sleeve body and/or at the distal end of the sleeve.

In some variants, the sleeve can be coupled with a balloon dilation device.

In some variants, the device can include a tip with the capability to hold an illuminating element for improved visualization during surgery.

In some variants, the illuminating element is a wire.

In some variants, the illuminating element is a fluorescent dye.

In some variants, the device can be placed over a variety of surgical instruments, conferring image guidance capabilities to instruments that were previously non-navigation enabled.

In some variants, a device for intranasal scanning of a patient's nose and sinuses is disclosed herein. The device can include a handle. The device can include a wand having image capture technology within the wand. The device can include a distal tip that can collect data. The distal tip can be placed into a passageway of a nasal sinus. The device can include a navigation-enabled component in the form of a wire, electrodes, and/or chip integrated into the distal tip. The device can include a light source equipped within the distal tip for illumination and/or transillumination of the sinuses or eustachian tubes.

In some variants, the shape of the distal tip may vary from flat, square to a blunt tip.

In some variants, the wand can be made of malleable and rigid materials to allow a person to configure the device to fit into various sinus ostia targeted for imaging.

In some variants, the handle can be designed to help prevent the device and surgeon's hands from abutting the patient's face.

In some variants, the distal tip can be detachable and can be attached to instruments that have been pre-bent or shaped.

In some variants, the device can communicate with CT scan systems to remodel pre-operative scans with intraoperative and postoperative anatomic images.

In some variants, a sinus stent for the treatment of a frontal sinus is disclosed herein. The stent can include an absorbable stent that can include alginate, collagen, connective tissue matrix, and/or chitosan honey-based leptospermum. The stent can include a three-layered structure, which can include two alginate and connective tissue matrix, and honey sheets and an alginate scaffolding interposed between the sheets, preventing adherence of secretions and crusting to the stent architecture. The stent can include a groove that can conform to a balloon sinus dilation delivery device for secure positioning. The absorbable stent can include a distal edge that can accommodate the anatomy of the frontal sinus drainage region.

In some variants, the stent can be composed of a combination of alginate, connective tissue matrix, honey, and collagen to maintain structural integrity for 2-4 weeks.

In some variants, the stent can be designed to resorb over the crucial postoperative time period.

In some variants, the combination of materials in the stent can confer the ability to be resorbed on a customizable time frame based on the frequency, volume, and chemical composition of irrigation solution utilized for postoperative debridement.

In some variants, the groove can secure the stent to the balloon sinus dilation delivery device, preventing it from dislodging during positioning prior to expansion.

In some variants, a suction irrigation device for the nasal and sinus cavities is disclosed herein. The device can include an irrigant chamber. The device can include a propulsion system. The device can include a malleable distal tip that can be bendable for targeting specific sinus regions. The device can include a suction port located inferior to the irrigation tip on the distal tip and shaft of the device.

In some variants, the distal tip can be detachable, allowing for the placement of different fixed angled distal tips.

In some variants, the irrigation tip can include a bivalved tip in combination with fluting along a set distance of the suction port.

In some variants, a superior portion of the device can contain a notch that can allow for attachment of an imaging device such as a rigid or flexible endoscope.

In some variants, an otologic device for treatment of an eustachian tube of a patient is disclosed herein. The device can include a straight portion. The device can include an angled portion extending away from a distal end of the straight portion. The angled portion can receive a sleeve thereon that is less rigid than the angled portion. A distal portion of the sleeve can be distal of a distal end of the angled portion. The distal portion can be disposed in the eustachian tube of a patient.

In some variants, the device can include a tapered section that can impede proximal movement of the sleeve past the tapered section.

In some variants, the tapered section can be disposed along the straight portion.

In some variants, the tapered section can decrease in diameter in a proximal-distal direction such that a distal portion of the otologic device comprises a diameter that is smaller than a diameter of a proximal portion of the otologic device.

In some variants, the straight portion can include a first portion and a second portion. The first portion can include a first diameter that is larger than a second diameter of the second portion.

In some variants, the straight portion can include a tapered section between the first portion and the second portion. The tapered section can increase in diameter size in the distal-proximal direction to impede proximal movement of the sleeve past the tapered section.

In some variants, the angled portion can extend away from the distal end of the straight portion at an angle.

In some variants, the angle can be 15, 20, 30, 40, 45, 50, or 60 degrees, and any values and ranges therein.

In some variants, the straight portion can include a first central longitudinal axis. The angled portion can include a second central longitudinal axis. The second central longitudinal axis can be oriented at an angle relative to the first central longitudinal axis.

In some variants, the angle can be 15, 20, 30, 40, 45, 50, or 60 degrees, and any values and ranges therein.

In some variants, the device can include an open proximal end, an open distal end, and an internal lumen connecting the open proximal end and the open distal end.

In some variants, the open proximal end can receive a medicament that flows through the internal lumen, out the open distal end, and into the distal portion of the sleeve.

In some variants, the distal portion of the sleeve can include perforations.

In some variants, the perforations can deliver medicament therethrough to the eustachian tube.

In some variants, the distal portion of the sleeve can include an expandable member that can dilate the eustachian tube.

In some variants, the expandable member can be a balloon.

In some variants, the distal portion of the sleeve can deliver a stent to the eustachian tube.

In some variants, the device can include a curve disposed at a junction between the angled portion and the straight portion.

In some variants, the device can include a handle.

In some variants, the handle can be ergonomic.

In some variants, the handle can be disposed at a handle angle relative to the straight portion.

In some variants, the handle can be disposed at a handle angle relative to a central longitudinal axis of the straight portion.

In some variants, the handle angle can be 10 or 15 degrees.

In some variants, the straight portion can be 5, 6, 7, 8, or 9 centimeters in length.

In some variants, the angled portion can be 2, 3, or 4 centimeters in length.

In some variants, the distal portion of the sleeve can be flexible to avoid damaging anatomy of the patient.

In some variants, the angled portion can direct positioning of the sleeve.

In some variants, the device comprises a cylindrical tube shape.

In some variants, the device can include the sleeve.

In some variants, a method of treating an eustachian tube of a patient is disclosed herein. The method can include disposing a flexible sleeve over a distal end of an otologic device. The method can include proximally advancing the flexible sleeve over an angled portion of the otologic device and a straight portion of the otologic device such that a distal portion of the flexible sleeve is distal of the distal end of the otologic device. The otologic device can include a tapered section that can increase a diameter of the otologic device in the distal-proximal direction to impede further proximal advancement of the flexible sleeve. The method can include inserting the flexible sleeve and the distal end of the otologic device through a nostril of a patient. The method can include navigating the distal portion of the flexible sleeve to the eustachian tube.

In some variants, the method can include expanding a balloon on the distal portion of the flexible sleeve to dilate the eustachian tube.

In some variants, the method can include delivering a stent on the distal portion of the flexible sleeve to the eustachian tube.

In some variants, the method can include flowing a medicament into an open proximal end of the otologic device, through an internal lumen of the otologic device, out the distal end of the otologic device, and into the distal portion of the flexible sleeve for delivery to the eustachian tube.

In some variants, the distal portion can include perforations configured to facilitate passage of medicament from inside the distal portion of the flexible sleeve to the eustachian tube.

Any feature, structure, or step disclosed among the embodiments herein can be replaced with or combined with any other feature, structure, or step disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

### BRIEF DESCRIPTION OF THE DRAWINGS

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure. For a fuller understanding of the nature of embodiments of the present invention, reference should be had to the following detailed description taken in connection with the accompanying drawings in which:

FIG. 1 is a schematic representation of the sinus sleeve device according to one embodiment, illustrating the incorporation of a distal dilation balloon and an proximally placed balloon for repeat dilation and stent placement, which can be slid over size-appropriate instruments.

FIG. 2 is a schematic representation of the proximal balloon in a collapsed and an expanded state, showcasing one or more grooves to assist in stent loading and prevent stent migration during the procedure.

FIG. 3 is a schematic representation of two separate inflation ports, allowing for individual inflation of the distal balloon and the proximal balloon.

FIG. 4 is a schematic representation of an embodiment of an endoscopic navigation system with a navigation wire or navigation electrode, a component that aids in precise localization and guidance during surgical procedures, an endoscope, a component for visualizing the surgical site and navigating through obstructed anatomy, a rigid but malleable sleeve housing the navigation wire or electrode and the endoscope, providing protection, support, and flexibility and an ergonomically designed handle that positions the surgeon's hand for enhanced control and reduced fatigue.

FIG. 5 is a schematic representation of the navigation-enabled endoscopic platform, serving as the base component for the endoscopic navigation device with an optional sinus dilation sleeve that can be slid onto the navigation-enabled endoscopic platform for sinus dilation without a separate balloon dilation apparatus.

FIG. 6 is a schematic representation of the device coupled with the optional sinus dilation sleeve, integrating a dilation balloon into the body of the endoscopic navigation system for an all-in-one solution during sinus and eustachian tube surgeries.

FIG. 7 is a schematic representation of the sleeve component, designed to be placed over size-appropriate surgical instruments with an attached balloon, aiding in sinus dilation, wire in the sleeve, providing image guidance navigation, and an electrode in the sleeve, offering image guidance navigation.

FIG. 8 is a schematic representation of the wire as an illuminating element at the tip of the device, confirming accurate anatomic placement and a fluorescent dye as an illuminating element at the tip of the device, verifying accurate anatomic placement according to one embodiment.

FIG. 9 is a schematic representation of an embodiment of navigation-enabled sleeve device, featuring a navigation-enabled sleeve without a balloon for dilation, a navigation wire within the body of the navigation-enabled sleeve, and a navigation electrode within the body of the navigation-enabled sleeve.

FIG. 10 is a schematic representation of an embodiment of a malleable wand with image capture technology embedded within the wand and its distal tip for collecting scan data.

FIG. 11 is a schematic representation of an embodiment of the detachable distal tip connected to the end of pre-bent instruments designed for optimal sinus imaging access.

FIG. 12 is a schematic representation of the device communicating with CT scan systems to combine pre-operative scans with intraoperative and/or postoperative anatomic findings/images, providing immediate intraoperative and in-office point-of-care information for the surgeon and patient.

FIG. 13 is a schematic representation of the sinus stent's three-layer structure, consisting of two alginate/honey sheets and an alginate scaffolding according to one embodiment.

FIG. 14 is a schematic representation of the cylindrical stent with an alginate framework interposed between the honey/alginate sheets according to one embodiment.

FIG. 15 is a schematic representation of the sinus stent's groove and shape, designed to conform to a balloon sinus dilation delivery device with an expanded sinus stent, illustrating its unique hourglass shape with a distal edge/flange that accommodates the complex anatomy of the frontal sinus drainage region according to one embodiment.

FIG. 16 is a schematic representation of the sinus stent's resorption process, which occurs on a customizable timeframe depending on the irrigation solution used for postoperative debridement.

FIG. 17 is a schematic representation of the sinus irrigation and suction device for the surgeon according to one embodiment.

FIG. 18 is a schematic representation of a detachable distal tip for the device and different fixed angled tips that can be attached to the device according to one embodiment.

FIG. 19 is a schematic representation of the superiorly placed notch for accommodating an endoscope or imaging device according to one embodiment.

FIG. 20 is a schematic representation of the inferiorly located suction port/tip in the distal tip of the device, a novel bivalved and fluted suction tip attached underneath the irrigation tip of the device and the suction mechanism for irrigation liquid in the event of distal tip obstruction according to one embodiment.

FIG. 21A is a schematic illustration of an otologic device to position a flexible sleeve in the eustachian tube.

FIG. 21B is a schematic illustration of the otologic device of FIG. 21A with a flexible sleeve disposed thereon.

FIG. 21C is a schematic illustration of the otologic device of FIG. 21A positioning a flexible sleeve in an eustachian tube.

FIG. 22A illustrates an otologic device with a flexible sleeve disposed thereon with a conduit connected to an expandable member disposed on the flexible sleeve.

FIG. 22B illustrates the otologic device of FIG. 22A with the flexible sleeve deflected.

### DETAILED DESCRIPTION

The term “sinusitis” refers generally to any inflammation or infection of the paranasal sinuses caused by bacteria, viruses, mold, allergies or combination of such factors. In more practical terms, it is estimated that chronic sinusitis results in millions of individuals visiting physician's offices on a yearly basis in the United States. Further, individuals suffering from sinusitis typically experience at least some symptoms including, but not limited to, headaches, facial pain, nasal congestion, difficulty in breathing and pain in the oral cavity. In situations where sinusitis cannot be successfully treated with antibiotics, decongestants and steroid nasal sprays, other procedures including catheter-based interventions are sometimes utilized. Accordingly, relief from such symptoms and causal factors includes restoring blocked mucus flow. As set forth above, various methods and devices for treatment of sinusitis and other conditions involving blocked mucus are known and include the dilating of the sinus ostia with various types of dilator devices. Typically, such devices are inserted trans-nasally or by a trans-canine fossa approach.

In various medical procedures including, but not limited to, the dilation of the paranasal sinus areas dilation devices, including balloon catheters, have been used. Expandable catheters have been available for many years. However, in recent years additional development has been directed to the utilization, application, and design of detachable catheters capable of being used in various medical procedures. In use, the catheter or other positioning instrument may be integrally associated with the expandable or inflatable devices designed and structured to be passed along a sometimes circuitous path to reach the site intended to be dilated.

The frontal sinus is considered the most anatomically intricate and surgically challenging among all nasal sinuses. This complexity arises due to the anatomy of the frontal sinus outflow tract. The frontal sinus is situated above the frontal beak of the frontal bones, with its outflow and drainage tract confined laterally by the orbits and the skull base. This configuration results in a restricted area for surgical dissection, consequently increasing the risk of major complications and stenosis. In addition to the inherent complexities of the surgical anatomy, both surgeons and patients face challenges in accessing the area for postoperative observation and wound care.

Chronic sinusitis is a prevalent medical condition affecting a significant portion of the global population. The treatment of chronic sinusitis often necessitates surgical intervention. Over the past 15 years, balloon sinus dilation has emerged as a well-established and widely employed surgical procedure for addressing chronic sinusitis. However, irrespective of the surgical approach utilized, stenosis and recurrent obstruction of the surgically dilated or opened sinuses remain frequent complications. Consequently, the insertion of a stent is a routine therapeutic strategy to mitigate the risk of restenosis.

A notable challenge in the context of balloon sinus dilation accompanied by stent placement is the multiplicity of procedural steps and device manipulation necessary to complete the operation. For example, the extant surgical methods and apparatuses obligate the surgeon to withdraw an accurately positioned dilation device from the operative field to load and deploy a stent. This requirement results in repeated tissue injury, hemorrhage, and diminished surgical efficiency. Accordingly, there is a need for an improved device that simplifies the procedure, reduces operating time, and optimizes the placement of stents for maintaining sinus patency.

Within the realm of medical and surgical practice, a significant void is evident in the availability of tools that facilitate comprehensive visualization of sinus anatomy and pathological conditions at various stages of surgical intervention. Specifically, the dearth of instruments designed for preoperative, intraoperative, and postoperative stages significantly hampers the ability to diagnose, monitor, and assess the progression or resolution of sinus ailments. This lack of appropriate visualization tools not only presents substantial challenges in delivering optimal care but also potentially compromises patient outcomes, due to potential missed or inaccurate diagnoses, suboptimal surgical interventions, and less effective postoperative monitoring. This glaring gap underscores the pressing need for the development and introduction of specialized tools capable of enhancing sinus visualization throughout the entire surgical process.

In the current state of surgical practice, there exists a deficiency in the provision of tools required by surgeons for the preoperative, intraoperative, and postoperative visualization of sinus anatomy and pathological conditions. Surgical instruments have not been specifically designed to assist surgeons treating refractory sinusitis and there is a lack of tools available to improve the postoperative care of initial and revision sinus surgeries. Accordingly, there is a need for devices that could assist and improve the whole medical procedure during the full course of a patient's treatment of sinus. Additionally, sinus surgery often requires the use of multiple instruments and devices, such as endoscopes, navigation systems, and dilation balloons. Conventional methods involve switching between these devices during surgery, which can be cumbersome and time-consuming. Accordingly, there is a need for an integrated device that combines these functionalities into one efficient, easy-to-use instrument.

In several embodiments, a delivery system is configured to automate via tooling and/or robotics to allow the medical practitioner to use both hands to control deployment, operation, dilation, inflation, and/or deflation of a balloon, stent, suction, irrigation, light or other feature.

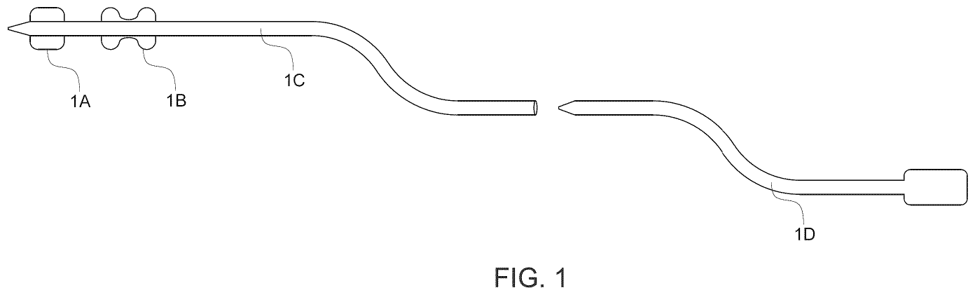

FIG. 1 provides a comprehensive view of an embodiment of the sinus sleeve design, which incorporates a distal dilation balloon 1A and an proximally placed balloon 1B, specifically engineered for repeat dilation and stent placement. This advanced arrangement ensures compatibility with size-appropriate instruments, allowing the sinus sleeve to slide smoothly during the procedure. In FIG. 1, an embodiment of the lead balloon 1A is illustrated, showcasing its primary function in expanding and opening the obstructed sinus ostia. The lead balloon's (1A) advanced material and design enable effective and precise dilation. Once the desired level of dilation is achieved, the lead balloon 1A can be easily deflated, allowing the device to progress to the subsequent stage, which involves positioning the second balloon 1B and stent for optimal placement. In one embodiment, the intricate design of the hourglass-shaped inferior balloon 1B is specifically engineered to facilitate the most advantageous placement of a stent within the frontal sinus drainage pathway. This unique shape ensures improved contact with the sinus walls, minimizing the risk of complications during the procedure. In various embodiments, an elongate member 1C of the sinus sleeve device is configured to slide over an elongate guide tool 1D.

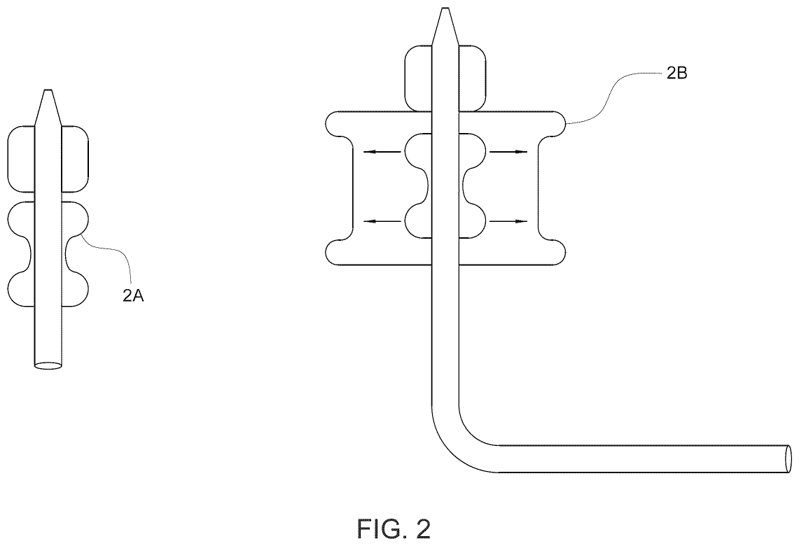

As demonstrated in FIG. 2, an embodiment of the proximal balloon in a collapsed state 2A incorporates one or more grooves that serve a dual purpose: assisting in stent loading and preventing any unwanted stent migration throughout the medical procedure. These grooves offer enhanced stability and support during stent deployment. the proximally located balloon is displayed in its expanded state 2B, which is achieved when the stent has been accurately positioned at the target anatomic location. This innovative design sequence significantly reduces the number of steps required for surgeons, eliminating the need for dilation, removal of the dilation device, stent loading, and reinsertion. FIG. 2 elaborates on this streamlined process, detailing how the surgeon can effectively dilate the obstructed sinus ostia using the lead, distal balloon, advance the device with the preloaded stent, and deploy the stent via the expansion of the second, proximal balloon. This efficient method minimizes the potential for complications and enhances overall surgical outcomes.

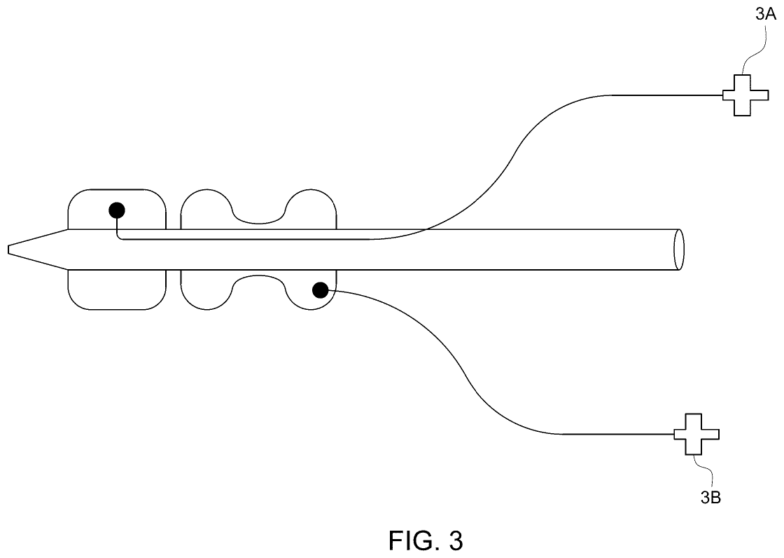

FIG. 3 demonstrates an embodiment with the inclusion of two separate inflation ports 3A, 3B in the sinus sleeve device. These ports give the surgeon the ability to individually inflate the distal balloon with a distal inflation port 3A and inflate the secondary, proximal stent balloon with a proximal inflation port 3B, promoting greater control and flexibility during the procedure. This innovative feature allows for sequential inflations, with or without a stent, resulting in more targeted dilation at the sinus outflow tract. Furthermore, it eliminates the need to reposition the device during the procedure, saving time and reducing the risk of complications.

Additionally, embodiments of the sinus sleeve's advanced material composition ensures flexibility, durability, and biocompatibility, minimizing any potential adverse reactions while providing long-lasting effectiveness. The device's ergonomic design further promotes case of use for the surgeon, enhancing overall procedural efficiency and accuracy. In summary, the advanced sinus sleeve device, as depicted in FIGS. 1 through 3, offers a comprehensive solution to streamline sinus procedures. By incorporating a lead, distal dilation balloon, an inferiorly placed, proximal balloon with an hourglass shape, separate inflation ports, and innovative features such as grooves for stent loading, the device significantly enhances surgical efficiency, precision, and overall patient outcomes.

In one embodiment, an endoscopic navigation system is designed to aid sinus and eustachian tube surgeons in visualizing, confirming location with navigation, and dilating obstructed anatomy using a single, one-handed device. This system aims to improve surgical efficiency by reducing the need to switch between multiple instruments, minimize tissue trauma through precise maneuvering, and alleviate surgeon fatigue by integrating multiple functionalities into a single ergonomic device.

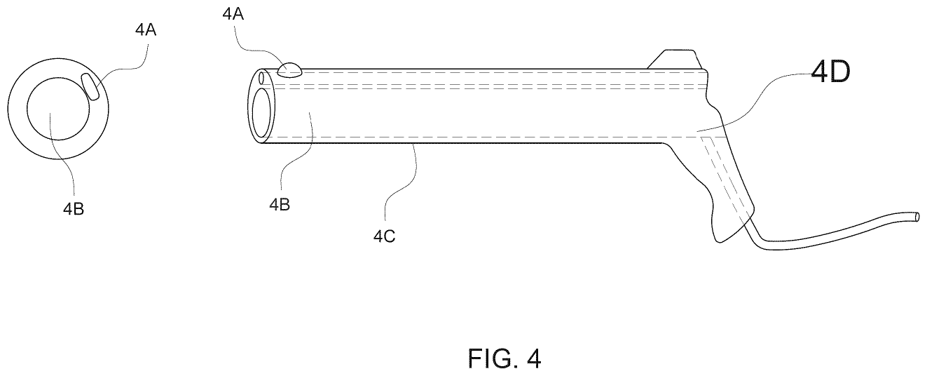

FIG. 4 is a schematic representation of an embodiment of an endoscopic navigation system with a navigation wire 4A or navigation electrode 4A, a component that aids in precise localization and guidance during surgical procedures, an endoscope 4B, a component for visualizing the surgical site and navigating through obstructed anatomy, a rigid but malleable sleeve 4C housing the navigation wire or electrode and the endoscope, providing protection, support, and flexibility and an ergonomically designed handle 4D that positions the surgeon's hand for enhanced control and reduced fatigue. Reference 4A illustrates an embodiment of the navigation wire or navigation electrode, as a component of the endoscopic navigation system, which helps in accurate localization and guidance during surgical procedures. This component ensures that the surgeon can safely and efficiently navigate through the patient's anatomy, minimizing the risk of complications and improving surgical outcomes. Reference 4B features an embodiment of the endoscope, another element of the system that enables the surgeon to visualize the surgical site and navigate through the obstructed anatomy. The endoscope provides high-quality imaging, allowing for real-time assessment of the surgical area and enhancing the surgeon's ability to identify and address any issues that may arise during the procedure. Reference 4C illustrates the rigid but malleable sleeve that houses both the navigation wire or electrode and the endoscope. This sleeve provides protection and support for the enclosed components while maintaining the flexibility needed for precise maneuvering. The sleeve's design ensures the device's durability, allowing it to withstand the rigors of surgical procedures while still offering the necessary range of motion for optimal navigation and visualization. Reference 4D highlights the ergonomically designed handle of the device. This handle positions the surgeon's hand in a mechanically advantageous way, allowing for enhanced control and reduced fatigue during surgical procedures. The handle is shaped to fit comfortably in the surgeon's hand, providing a secure grip and promoting precise movements, which contribute to overall surgical success.

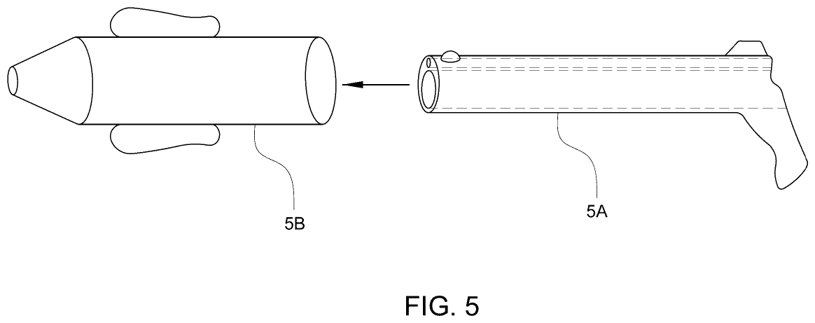

FIG. 5 is a schematic representation of the navigation-enabled endoscopic platform, serving as the base component for the endoscopic navigation device with an optional sinus dilation sleeve that can be slid onto the navigation-enabled endoscopic platform for sinus dilation without a separate balloon dilation apparatus. Reference 5a presents an embodiment of the navigation-enabled endoscopic platform, which serves as the base system for the endoscopic navigation device. This platform incorporates both the endoscope and the navigation wire or electrode, streamlining the surgical process by consolidating these essential components into a single, easy-to-use system. Reference 5B demonstrates the optional sinus dilation sleeve that can be slid onto the navigation-enabled endoscopic platform, providing a means for sinus dilation without the need for a separate balloon dilation apparatus. This feature increases the versatility of the device, enabling the surgeon to perform dilation procedures more efficiently and with fewer instrument exchanges.

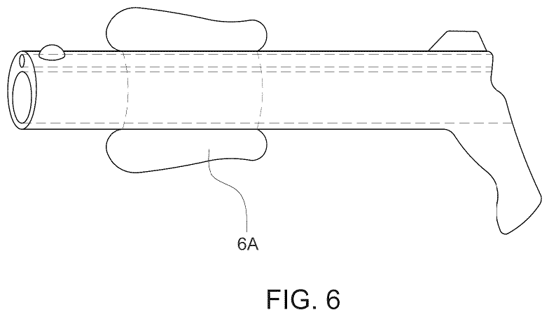

FIG. 6 demonstrates an embodiment of the device in which a dilation balloon is integrated into the body of the endoscopic navigation system, offering an all-in-one solution for visualization, navigation, and dilation during sinus and eustachian tube surgeries. This integrated design simplifies the surgical process by eliminating the need for additional tools, resulting in a more streamlined and efficient procedure that ultimately benefits both the surgeon and the patient. Reference 6A is a schematic representation of the dilation balloon mounted on the optional sinus dilation sleeve, employed for the expansion and opening of the obstructed sinus ostia. The dilation balloon can be attached to the sleeve. Accordingly, this embodiment provides an endoscopic and navigation-enabled device for sinus surgery that combines image guidance navigation and endoscopy in a single handheld instrument. The integration of a dilation balloon, ergonomic handle design, and navigation technology at the distal tip make this device a more efficient and user-friendly solution for sinus surgery procedures.

In one embodiment, an innovative NavSleeve and Tip apparatus addresses the challenges associated with sinus dilation and navigation instrumentation, particularly in minimizing invasiveness and increasing accuracy during the procedure.

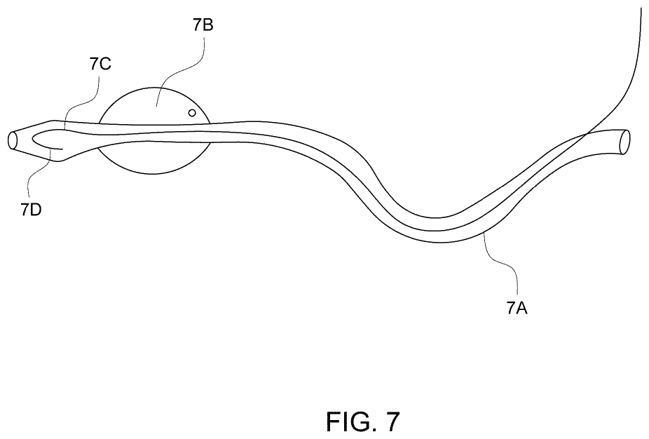

FIG. 7 is a schematic representation of the sleeve component, designed to be placed over size-appropriate surgical instruments with an attached balloon, aiding in sinus dilation, wire in the sleeve, providing image guidance navigation, and an electrode in the sleeve, offering image guidance navigation. In various embodiments, the device includes a navigation interface. The navigation interface may be at least partially defined as an external device that may be located on the exterior of the body, which allows for real-time tracking without needing to enter the patient's body. For example, the external device may be located on the face of a person, and in substantial alignment with the paranasal sinuses. The external device may be configured to substantially duplicate the movement of the dilator device by forming an at least partially operable magnetic interface with the tip of the sleeve. Accordingly, the external device may comprise a magnet while the tip may comprise a material capable of being attracted to the magnet, such as a ferromagnetic material. The magnetic interface may be used to determine or track at least an approximate position of the dilator device within the paranasal sinuses, improving the safety and accuracy of the procedure. The navigation interface may also be at least partially defined as a component observable on a variety of imaging capabilities. The tip may comprise a material that may act as a contrast agent on the particular imaging capability, enhancing visibility and aiding the surgeon. For example, the tip may comprise barium sulfate which is a contrast agent that is viewable on an x-ray.

Reference 7A illustrates an embodiment of the sleeve component of the device, which is intended to be placed over size-appropriate surgical instruments, making it adaptable for use with various tools. Reference 7B shows the attached balloon, which aids in sinus dilation, a crucial feature for effectively treating sinus issues such as chronic sinusitis or nasal polyps. The device features a navigation component that provides image guidance navigation through the body of the sleeve, leading to the tip of the sinus dilation sleeve. This is achieved by incorporating a wire or electrode in the sleeve and/or tip, as depicted in Reference 7C and Reference 7D. This design allows for the conversion of non-navigation instruments into navigation-enabled instruments, reducing assembly time and the learning curve associated with balloon sinus dilation equipment, making the procedure more accessible for medical professionals. Furthermore, this solution enables facilities to avoid replacing expensive navigation equipment that has been bent, distorted, or has lost its image guidance function and accuracy. Instead, a navigation dilation sleeve can be placed over the device to restore accurate image-guided navigation, promoting cost-effectiveness and reducing downtime.

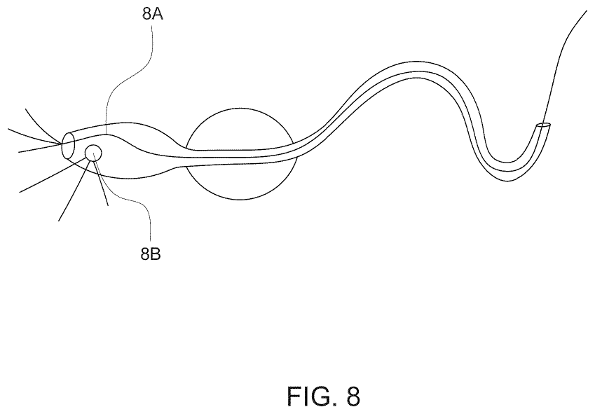

FIG. 8 is a schematic representation of the wire as an illuminating element at the tip of the device, confirming accurate anatomic placement and a fluorescent dye as an illuminating element at the tip of the device, verifying accurate anatomic placement according to one embodiment. To enhance the accuracy and functionality of the sinus navigation dilation sleeve, several additional features are proposed. For instance, the tip of the device may hold an illuminating element, such as a wire (Reference 8A) or fluorescent dye (Reference 8B), providing an additional signal to confirm accurate anatomic placement of the device, ensuring that the procedure is carried out precisely and minimizing the risk of complications.

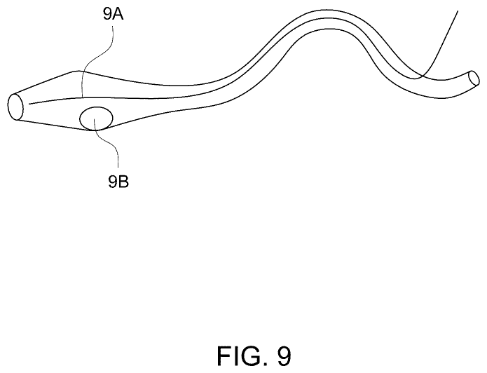

In one embodiment, the device shown in FIG. 9 includes a navigation-enabled sleeve without a balloon for dilation. This navigation sleeve imparts image guidance capabilities to a variety of surgical instruments, making it versatile for use in different surgical applications. The sleeve contains either a navigation wire (Reference 9A) or navigation electrode (Reference 9B) within its body, enabling compatibility with different navigation systems and enhancing the device's utility in various surgical settings.

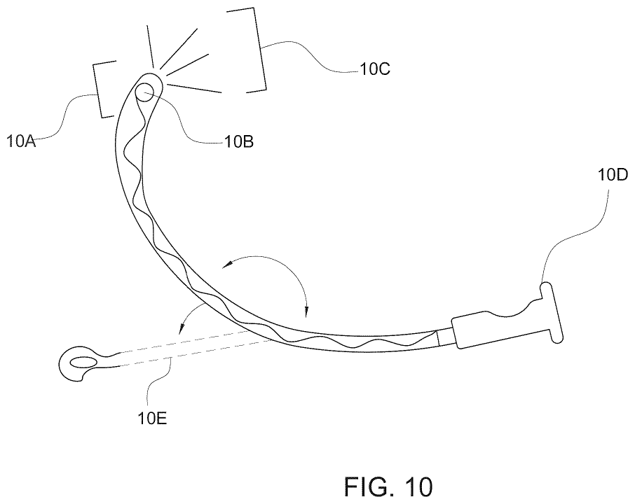

In various embodiments, an intranasal scanner device comprises a malleable wand with image capture technology embedded within the wand and its distal tip for collecting scan data, as illustrated in FIG. 10. The wand and tip can incorporate various imaging technologies, including triangulation, parallel confocal imaging, accordion fringe interferometry, or three-dimensional in-motion video, providing flexibility and adaptability to different imaging needs. Designed to facilitate access to narrow nasal sinus passageways, the thin and narrow tip may come in various shapes, such as flat, square, or blunt, to aid in imaging sinus ostia (Reference 10A). These diverse tip shapes enhance the device's capability to navigate and obtain accurate images of the complex sinus anatomy.

In one embodiment, the distal tip accommodates navigation-enabled instrumentation in the form of a wire, electrodes, and/or chip (Reference 10B). This compatibility with pre-operative navigation-enabled CT scans supports device placement during intraoperative and/or postoperative reconstruction, increasing the accuracy of surgical procedures. Additionally, the distal tip is equipped with a light for transillumination of the sinuses, assisting in confirming the camera tip's placement in the targeted sinus ostia (Reference 10C). This feature enhances the surgeon's ability to accurately visualize the sinus anatomy during the procedure. The handle of the device is designed for easy placement into the nose and sinuses, promoting proper ergonomics and preventing the device and surgeon's hands from touching the patient's face (Reference 10D), thereby maintaining sterility and patient comfort. The device's malleability allows the surgeon to configure it for specific sinus ostia imaging, such as gently curving it for frontal sinus imaging and then straightening it for ethmoid and sphenoid sinus imaging (Reference 10E). A 90-110-degree bend can be made for maxillary sinus imaging. This adaptability ensures that the device can effectively access and image various sinus regions, catering to individual patient anatomies and specific surgical requirements. Reference 10A is a schematic representation of the various shapes of the distal tip designed to facilitate access to narrow nasal sinus passageways and aid in imaging sinus ostia. Reference 10B is a schematic representation of the distal tip accommodating navigation-enabled instrumentation in the form of a wire, electrodes, and/or chip. Reference 10C is a schematic representation of the distal tip equipped with a light for transillumination of the sinuses, assisting in confirming the camera tip's placement in the targeted sinus ostia. Reference 10D is a schematic representation of the ergonomically designed handle for easy placement into the nose and sinuses while preventing the device and surgeon's hands from touching the patient's face. Reference 10E is a schematic representation of the device's malleability, allowing the surgeon to configure it for specific sinus ostia imaging.

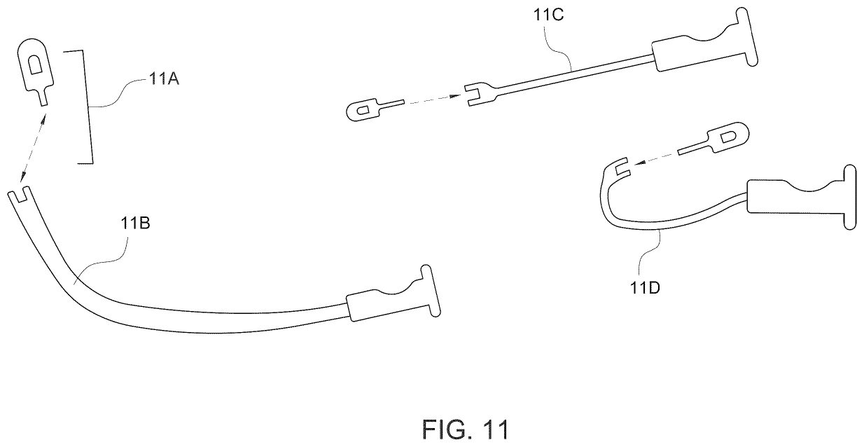

In one embodiment, FIG. 11 illustrates the detachable distal tip can be connected to the end of pre-bent instruments, designed for optimal sinus imaging access (Reference 11A). These instruments may be reusable or disposable (Reference 11B, Reference 11C, and Reference 11D), providing flexibility in terms of surgical instrument selection and facilitating adherence to institutional protocols or budgetary constraints. Reference 11A is a schematic representation of the detachable distal tip connected to the end of pre-bent instruments. Reference 11B, Reference 11C, and Reference 11D are schematic representations of the reusable or disposable pre-bent instruments designed for optimal sinus imaging access.

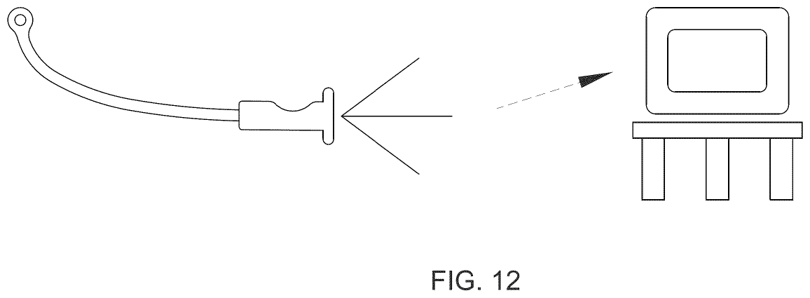

In one embodiment, the device communicates with CT scan systems to combine pre-operative scans with intraoperative and/or postoperative anatomic findings/images, eliminating the need for a full repeat CT scan and reducing radiation exposure for the patient. This provides immediate intraoperative and in-office point-of-care information for the surgeon and patient (FIG. 12), enhancing surgical decision-making and patient care. Data transmission may occur via a hardwired connection or Wi-Fi, allowing for seamless integration with existing medical imaging infrastructure and improving the overall efficiency of the surgical process.

In one embodiment, the intranasal/sinus scanner projects a light source, such as a laser or structured light, onto the sinus ostia to be scanned. Imaging sensors capture the images of the sinus ostia, and the scanning software processes these images to generate point clouds. The software then triangulates the point clouds to create a 3D surface model (mesh) of the sinus anatomy. This digital intranasal 3D reconstruction serves as a ‘virtual’ alternative to traditional methods, providing a more accurate representation of the sinus ostia during and after surgery. The objective of this scanner is to facilitate a more accurate assessment of successful sinus surgery and postoperative surveillance of ostia patency. By providing early detection of the need for further treatment, the device enhances patient care and optimizes surgical outcomes. The intranasal/sinus scanner can be integrated with existing medical imaging systems and surgical tools, offering seamless compatibility and adaptability in various clinical settings. The device's non-invasive nature and real-time imaging capabilities enable its application in both intraoperative and postoperative scenarios, promoting effective surveillance and follow-up for sinus surgery patients.

In one embodiment, frontal sinus stents and spacers, such as the sinus stent, can be used intraoperatively or postoperatively. These devices aim to maintain separation of wound edges to prevent synechia formation, occupy dead space to prevent the collection of secretions, and potentially serve as a matrix for mucosalization. The sinus stent is an absorbable stent/spacer comprising a percentage (e.g., 5%, 10%, 20%, 25%, 30%, 40%, 50%, 60%, 70%, 75%, 80%, 90%, 95% and values and ranges therein) of alginate, collagen, connective tissue matrix, or chitosan and a percentage (e.g., 5%, 10%, 20%, 25%, 30%, 40%, 50%, 60%, 70%, 75%, 80%, 90%, 95% and values and ranges therein) of honey-based leptospermum. It has several unique features designed for use in the frontal sinus.

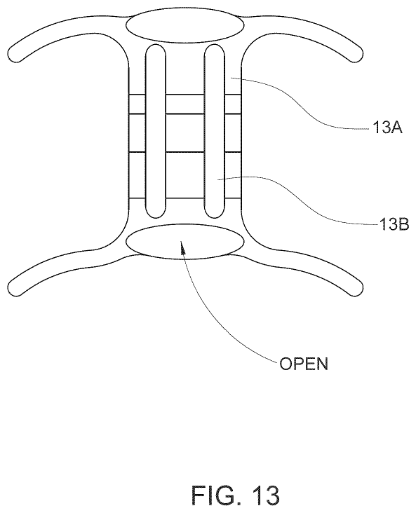

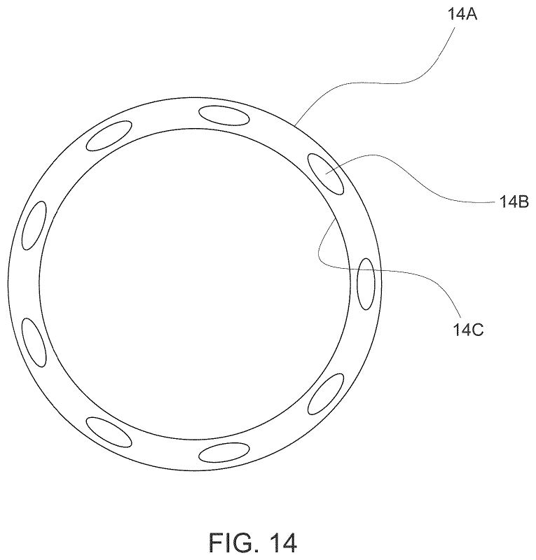

In one embodiment illustrated at FIG. 13, the stent comprises of three layers: two alginate/honey sheets, whose shape and design are maintained by a scaffolding of a certain percentage of alginate, collagen, connective tissue matrix, or chitosan (Reference 13A). This results in a cylindrical stent with an alginate framework interposed between the honey/alginate sheets (Reference 14A, Reference 14B, and Reference 14C). This structure prevents the adherence of secretions and crusting to the stent architecture, reducing synechiae formation and subsequent sinus outflow tract stenosis.

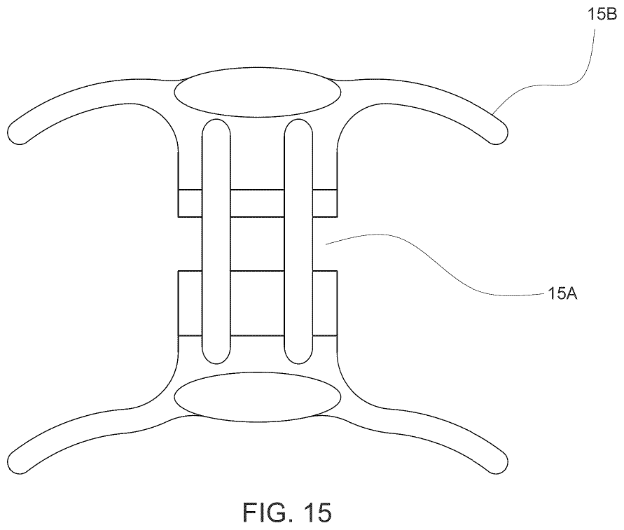

The sinus stent embodiment in FIG. 15 features a groove and is shaped to conform to a balloon sinus dilation delivery device. This design allows the stent to adhere more securely to the balloon, preventing dislodgement during positioning before expansion (Reference 15A). Moreover, once expanded, the stent/spacer maintains a unique hourglass shape with a distal edge/flange that accommodates the complex anatomy of the frontal sinus drainage region (Reference 15B).

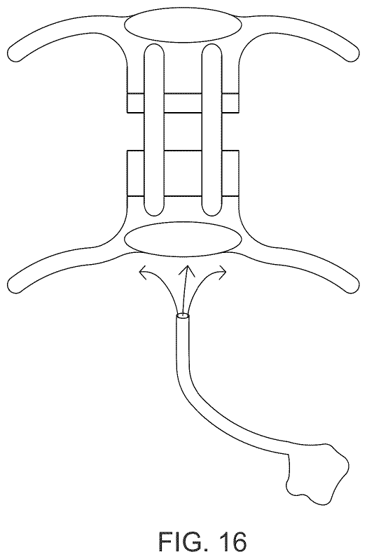

The stent is designed with a percentage (e.g., 5%, 10%, 20%, 25%, 30%, 40%, 50%, 60%, 70%, 75%, 80%, 90%, 95% and values and ranges therein) of various materials, such as alginate, collagen, connective tissue matrix, chitosan, and/or honey to maintain structural integrity for 2-4 weeks. The stent begins to resorb during this critical postoperative period (FIG. 16). The combination of these materials also endows the stent with the ability to resorb on a customizable timeframe, depending on the frequency, volume, and chemical composition of the irrigation solution used for postoperative debridement. There is potential to add an ingredient, such as iodine, to the irrigation, which accelerates the resorption of the stent. This could be delivered by a sinus irrigation device.

Additional features found in various embodiments of the sinus irrigator include the dilator device comprising an irrigation structure configured to provide irrigation to an intended body part. The tip may comprise an irrigation portion having at least one opening disposed in fluid communication with at least a portion of the hollow interior and configured to provide irrigation. Consequently, a fluid may pass through the hollow interior and exit through the irrigation opening(s) to irrigate the intended body part.

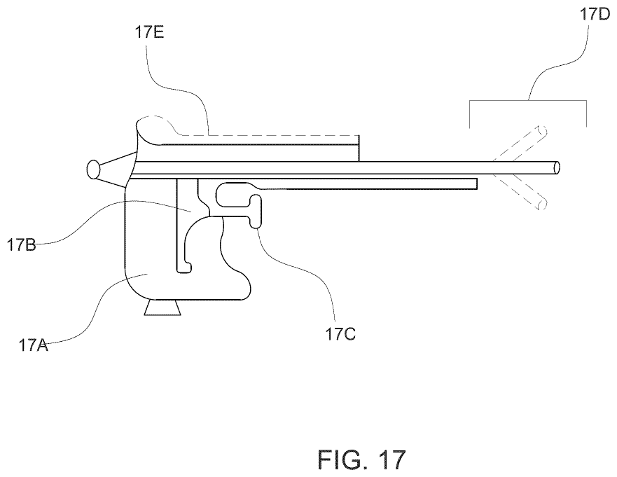

Surgical treatment of the sinuses often involves functional endoscopic sinus surgery and/or balloon sinus dilation, leading to the postoperative formation of crusting in the operated sinus ostia and cavities. In response to this issue, FIG. 17 illustrates a sinus irrigation and suction device for the surgeon. The device comprises a chamber to hold sinus irrigant (Reference 17A) and a propulsion system (Reference 17B) actuated by the surgeon's finger (Reference 17C). A malleable distal tip (Reference 17D) allows the surgeon to adjust the bend for the targeted sinus cavity during irrigation. Reference 17A is a schematic representation of the chamber designed to hold sinus irrigant in the device. Reference 17B is a schematic representation of the propulsion system integrated into the device. Reference 17C is a schematic representation of the surgeon's finger actuating the propulsion system. Reference 17D is a schematic representation of the malleable distal tip for adjusting the bend during sinus cavity irrigation. Reference 17E is a schematic representation of the superiorly placed notch for accommodating an endoscope or imaging device.

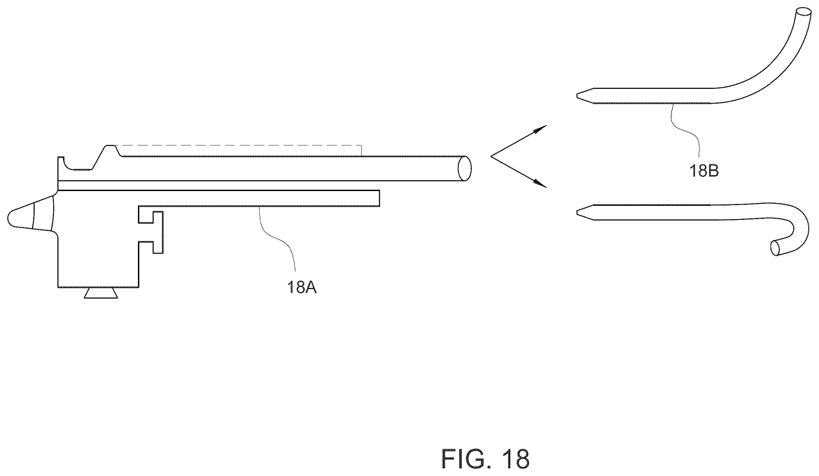

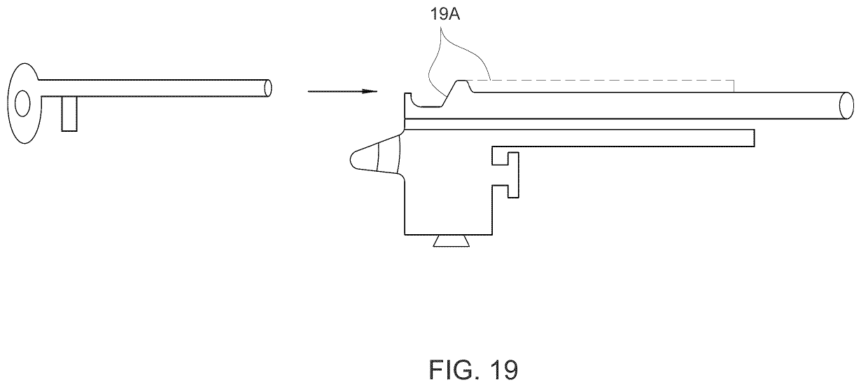

In one embodiment illustrated at FIG. 18, the distal tip may be detachable, enabling the attachment of different fixed angled tips to the device (Reference 18A and Reference 18B). Additionally, the irrigation and suction device features a superiorly placed notch to accommodate an endoscope or imaging device, such as a rigid or flexible endoscope (Reference 19A). This design allows the surgeon to maneuver the device with one hand, freeing the other hand for using another instrument to assist with sinus debridement, cleaning, and irrigation.

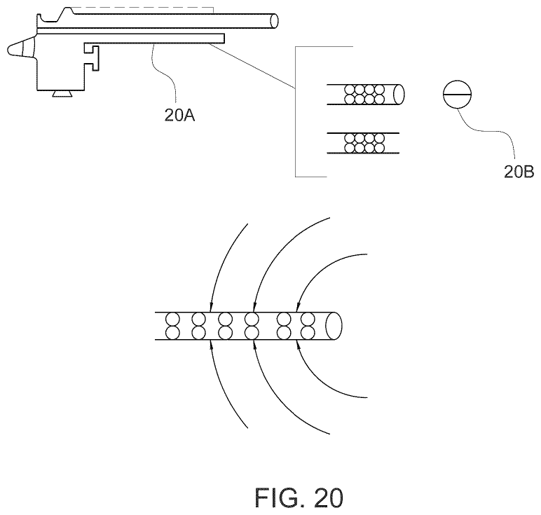

The distal tip includes an inferiorly located suction port/tip (Reference 20A). A crucial aspect of this device is the mechanism for preventing obstruction by crust and mucus, ensuring continuous suction. This is achieved through a novel bivalved and fluted suction tip (FIG. 20A and FIG. 20B) attached underneath the irrigation tip of the device. The bivalve design helps prevent distal tip obstruction, while the combination of fluting and the bivalve suction tip enables suction of irrigation liquid in the event of distal tip obstruction (FIG. 20C). This feature is essential for preventing the drainage of irrigant into the patient's posterior nasal cavity and avoiding aspiration.

The sinus irrigator, a disposable and cost-efficient device, facilitates single-handed postoperative care and cleansing of sinus cavities, thereby negating the necessity for additional assistance during sinus irrigation. Its inherent design promotes enhanced postoperative sinus healing and reduces the risk of scarring or synechiae formation. Moreover, the sinus irrigator is designed to function synergistically with the sinustent, thereby endowing the sinus surgeon with a diverse range of irrigation options. This versatility enables a tailored approach based on the surgeon's preference and the specific treatment requirements, further optimizing the outcome of the sinus surgical intervention.

### Otologic Devices and Methods

The eustachian tube is a vital anatomical structure connecting the middle ear to the nasopharynx. The eustachian tube extends from the anterior wall of the middle ear to the lateral wall of the nasopharynx. Anatomically, the eustachian tube includes a bony or osseous portion which lies within the petrous portion of the temporal bone and is continuous with the anterior wall of the middle ear. The eustachian tube also includes a fibrocartilaginous tube which courses anteromedially and inferiorly from the bony portion in a 40-45 degree plane until reaching the nasopharynx. The opening of the eustachian tube into the nasopharynx forms a prominence known as the torus tubarius. The typical length of the bony and cartilaginous eustachian tube measures 30-40 mm. The length of the cartilaginous portion ranges from 23-27 mm. The junction of the osseous and cartilaginous components form the narrowest portion of the eustachian tube known as the isthmus. The internal carotid artery passes near the eustachian tube. In several embodiments, a eustachian tube delivery system includes a delivery device that extends up to 2 cm (e.g., 0.5 cm, 1 cm, 1.5 cm, and 2 cm, 0.5-2 cm, 1-2 cm, 1.5-2 cm, and values and ranges therein) into a eustachian tube to reduce the likelihood of damaging the eustachian tube and/or anatomical features near the eustachian tube, such as surrounding bone, cartilage, tissue, and/or vascular such as the carotid artery.

In a healthy eustachian tube, the bony portion is open at all times and the cartilaginous end is closed at rest but opens during swallowing, yawning, chewing, or when forced open such as when performing a valsalva maneuver. The eustachian tube functions to equalize the pressure between the middle ear and outside environment and facilitate drainage of mucus secreted by the middle ear mucosa. Multiple conditions may result in impairment of these physiologic functions of the eustachian tube. These may include processes such as allergic rhinitis or sinusitis producing inflammation of the mucosal lining or neoplastic growths which physically impair the opening of the eustachian tube. The inability of the eustachian tube to perform its physiologic functions results in a condition termed eustachian tube dysfunction.

Eustachian tube dysfunction may result in a variety of pathologic conditions. These include acute and chronic ear infections, tympanic membrane retraction and perforation, and cholesteatoma formation. Symptoms associated with these disease states include hearing loss, ear fullness, ear pain, and/or tinnitus. Eustachian tube dysfunction is more common in children; however, adult eustachian dysfunction still has a prevalence of 4.6%. Eustachian tube dysfunction has been reported to account for over 2 million health care visits annually.

For many years, the treatment of eustachian tube dysfunction has been limited to medical therapy with systemic and topical steroids, antihistamines, systemic and topical decongestants. Failure of medical therapy has generally required surgical treatment for eustachian tube dysfunction with a myringotomy and placement of a pressure equalization tube (also known as a tympanostomy tube), which can result in a permanent tympanic membrane perforation and possible scarring or tympanosclerosis. Accordingly, devices and methods to facilitate surgical treatment for eustachian tube dysfunction that reduce or entirely eliminate damage to the anatomy (e.g., otologic anatomy) of a patient are desired.

The otologic devices and methods described herein facilitate surgical treatment for eustachian tube dysfunction with reduced or no damage to the anatomy (e.g., otologic anatomy) of a patient. The otologic device can include a tube with a lumen, a straight portion and an angled portion extending at an angle from an end of the straight portion. A flexible sleeve can be disposed over the angled portion and advanced proximally to a tapered section of the straight portion that impedes proximal advancement of the flexible sleeve such that a distal portion of the flexible sleeve is disposed distal of a distal end of the tube and maintains its flexibility. The distal portion of the flexible sleeve can be referred to as the overhanging flexible sleeve portion or overhanging sleeve. In several embodiments, a balloon is configured to extend from the angled portion, wherein the angled portion is inserted into an eustachian tube by no more than 2 cm (e.g., 0.5, 1.0, 15, 2.0 cm). In one embodiment, the lumen is configured to allow air or fluids to exit the eustachian tube to reduce pressure in the eustachian tube.

The distal portion of the flexible sleeve can be navigated to the eustachian tube for treatments. The soft, flexible sleeve can facilitate treatments and avoid damage to a patient's anatomy (e.g., otologic anatomy) due to its flexible characteristics. The distal portion of the flexible sleeve can provide a directed and angled but soft, flexible treatment sleeve into the eustachian tube. As the distal portion of the flexible sleeve contacts anatomy, the distal portion of the flexible portion can flex to avoid damage that may be caused by a more rigid device. The otologic device with the flexible sleeve can position an expandable member, such as a balloon, in the eustachian tube for dilation. The otologic device with the flexible sleeve can position a stent in the eustachian tube. The otologic device can facilitate the introduction of medicaments (e.g., antibiotics, anti-inflammatories, surfactants, anesthetics, and/or others) to the eustachian tube with the flexible sleeve. For example, the flexible sleeve can include perforations on the distal portion, which can facilitate the delivery of a medicament flowing into an open proximal end of the tube, out the open distal end of the tube, and into the flexible sleeve.

The otologic device can include a handle. The handle may be grasped by a clinician to navigate the tube and flexible sleeve through the anatomy of a patient. The handle may be angled relative to the straight portion, which can provide an ergonomically favorable hand position.

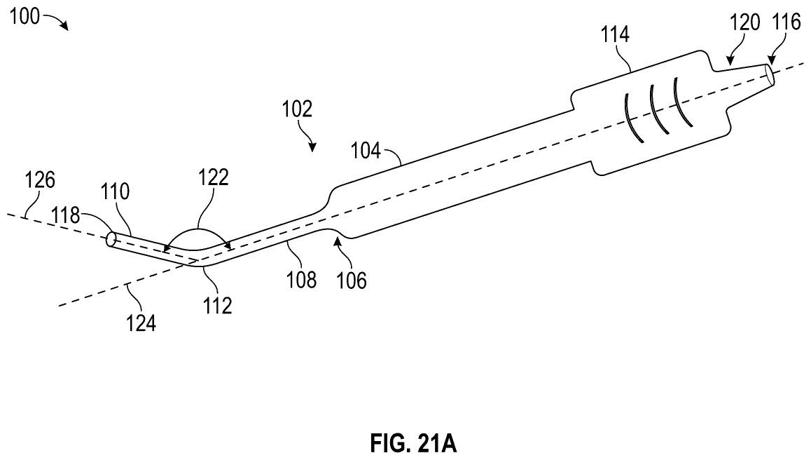

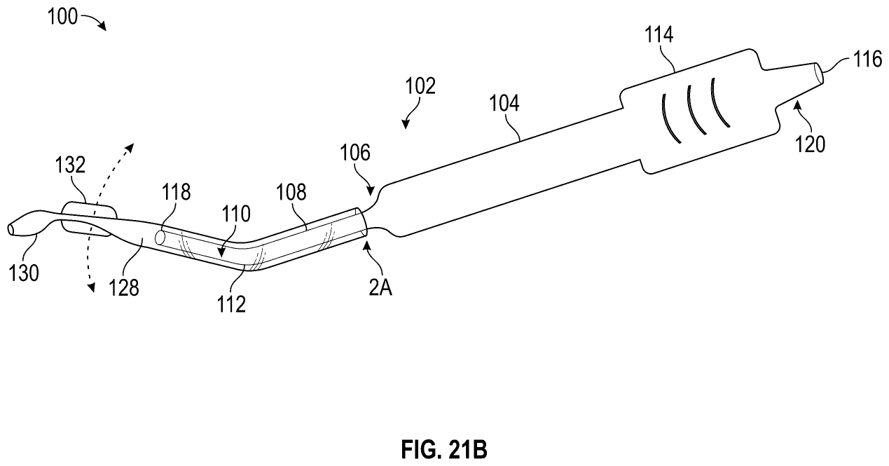

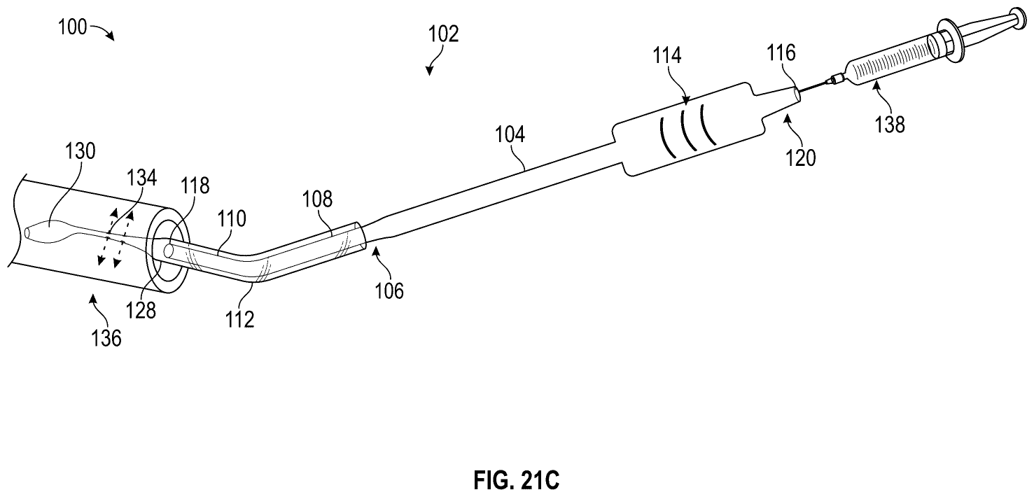

FIGS. 21A-21C schematically illustrate an example an example otologic device 100, which may be referred to as a device. As shown in FIG. 21A, the otologic device 100 can include a tube shape, which may be described as a conduit and/or lumen. The tube can be generally cylindrical. The otologic device 100 can include a straight portion 102. The straight portion 102 can have varying lengths, which can at least include less than 3, 3, 4, 5, 6, 7, 8, 9, 10, or more than 10 centimeters or any value between any of the foregoing. The straight portion 102 can include a first portion 104 and a second portion 108. The first portion 104 can have a first diameter. The second portion 108 can have a second diameter, which may be smaller than the first diameter of the first portion 104. A tapered section 106 can be disposed between the first portion 104 and second portion 108. The tapered section 106 can gradually, which may include consistently, reduce the diameter of the straight portion 102 from the first diameter of the first portion 104 to the second diameter of the second portion 108.

The otologic device 100 can include an angled portion 110. The angled portion 110 can extend from a distal end of the straight portion 102 (e.g., second portion 108 of the straight portion 102) at an angle relative to the straight portion 102. For example, the angle between the straight portion 102 and the angled portion 110 can be less than 20, 20, 30, 40, 50, or more than 50 degrees or any value between any of the foregoing. The straight portion 102 can include a first central longitudinal axis 124, and the angled portion 110 can include a second central longitudinal axis 126. The angle between the first central longitudinal axis 124 of the straight portion 102 and the second central longitudinal axis 126 of the angled portion 110 can be less than 20, 20, 30, 40, 50, or more than 50 degrees or any value between any of the foregoing. The otologic device 100 can include a curve 112 at the junction between the straight portion 102 (e.g., second portion 108 of the straight portion 102) and the angled portion 110. The otologic device 100 can curve from the straight portion 102 to the angled portion 110. The angled portion 110 can be varying lengths, which can be less than 0.5, 0.5, 1, 2, 3, 4, 5, 6, or more than 6 centimeters or any value between any of the foregoing.

The otologic device 100 can include a handle 114. The handle 114 can be disposed at a proximal position on the otologic device 100. The handle 114 can be disposed at an ergonomically favorable position for a clinician using the otologic device 100. For example, the handle 114 can be disposed at an angle relative to the straight portion 102. The handle 114 can be disposed at an angle relative to the first central longitudinal axis 124 of the straight portion 102, which can include less than 5, 5, 10, 15, 20, or more than 20 degrees or any value between any of the foregoing. In some variants, the handle 114 is not angled relative to the straight portion 102.

The otologic device 100 can include a proximal end 116 and a distal end 118. The proximal end 116 can be open. The distal end 118 can be open. An internal lumen can fluidically connect the proximal end 116 and the distal end 118, which can enable devices and/or medicaments introduced into the otologic device 100 at the proximal end 116 exit the otologic device 100 at the distal end 118. The otologic device 100 can include a tapered portion 120 disposed proximate the proximal end 116. The tapered portion 120 can decrease in diameter in the proximal direction, which can include decreasing in diameter until the proximal end 116. The tapered portion 120 can be disposed between the handle 114 and the proximal end 116. The tapered portion 120 can extend between the handle 114 and the proximal end 116.

As illustrated in FIG. 21B, the otologic device 100 can include a sleeve 128 disposed thereon, which may be described as a flexible sleeve, tube, and/or flexible tube. The sleeve 128 can be flexible. The sleeve 128 can be disposed over a distal portion of the otologic device 100. For example, the sleeve 128 can be disposed over the distal end 118 of the otologic device 100. The sleeve 128 can be disposed over the distal end 118 and advanced proximally over the angled portion 110 and/or second portion 108 of the straight portion 102. The tapered section 106 of the straight portion 102 can impede proximal movement of the sleeve 128. The sleeve 128 can be proximally advanced to the tapered section 106. The sleeve 128 can include a distal portion 130 distal of the distal end 118 of the otologic device 100. The distal portion 130 can extend away from the distal end 118 of the otologic device 100. The distal portion 130 can extend away from the distal end 118 of the otologic device 100 at the angle of the angled portion 110. The angled portion 110 can direct the position of the distal portion 130. The distal portion 130 of the sleeve 128 can maintain its flexible characteristics because the distal portion 130 is not disposed on the more rigid structure of the otologic device 100. The distal portion 130 of the sleeve 128 can maintain its flexibility because the distal portion 130 is disposed beyond the distal end 118 of the otologic device 100. The sleeve 128 can include an open proximal end and/or an open distal end, which can be connected by way of an internal lumen. The sleeve 128 can include an open proximal end disposed over the otologic device 100 and a closed distal end. The distal portion 130 can include a taper. The distal portion 130 can include a nozzle.

The sleeve 128 can include an expandable member 132, which can include a balloon. The expandable member 132 can be disposed around a periphery of the sleeve 128. A conduit can be routed through the internal lumen of the otologic device 100 to the expandable member 132 to supply a gas or liquid to the expandable member 132 for inflation. In some variants, a conduit can be disposed along the exterior of the otologic device 100 to the expandable member 132 to supply a gas or liquid. In use, the otologic device 100 can position the distal portion 130 of the sleeve 128 with the expandable member 132 in the eustachian tube. As described herein, the flexibility of the distal portion 130 of the sleeve 128 can avoid damage to tissue. With the expandable member 132 positioned in the eustachian tube, the expandable member 132 can be expanded to dilate the eustachian tube.

As illustrated in FIG. 1C, the otologic device 100 can be used to position the sleeve 128 in the eustachian tube 136 for medicament delivery. The distal portion 130 of the sleeve 128 can include one or more apertures 134, which can be referred to as perforations. The one or more apertures 134 can be used to delivery medicament inside the eustachian tube 136. For example, a medicament delivery device 138 (e.g., syringe) can deliver a medicament (e.g., antibiotics, anti-inflammatories, surfactants, anesthetics, and/or others) into the otologic device 100 by way of the proximal end 116. The medicament can flow through the internal lumen of the otologic device 100, out the distal end 118 of the otologic device 100, and into the distal portion 130 of the sleeve 128. The medicament can flow out of the distal portion 130 of the sleeve 128 by way of the apertures 134 and into the eustachian tube 136. In some variants, the distal end of the sleeve 128 can be open, enabling medicament to flow out of the open distal end of the sleeve 128.

As described herein, the sleeve 128 can be used to position a stent in the eustachian tube. The sleeve 128 with a stent disposed thereon can be navigated to the eustachian tube. The stent can be positioned in the eustachian tube with the sleeve 128 and then proximally retracted for removal.

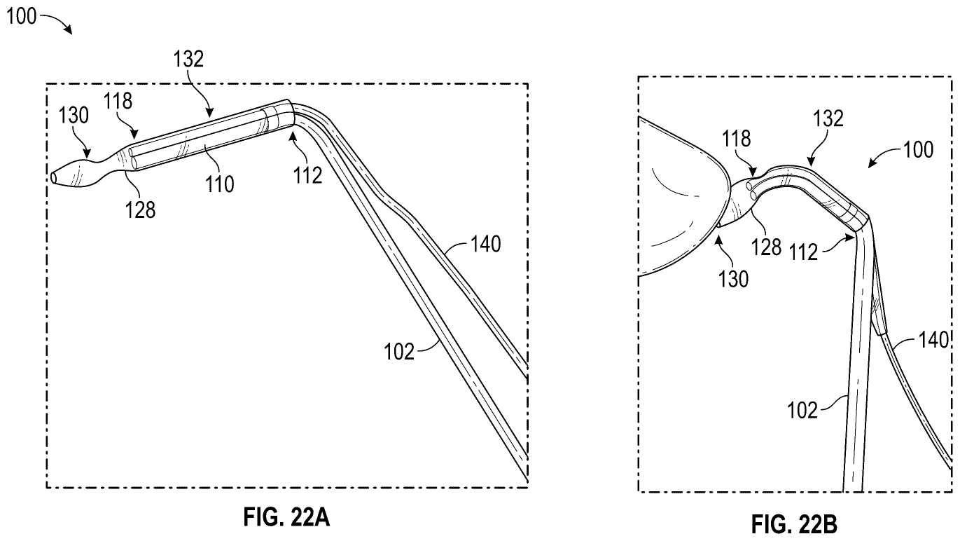

FIGS. 22A and 22B illustrate the otologic device 100. As illustrated, the otologic device 100 can include straight portion 102. The straight portion 102 can curve at the curve 112 to the angled portion 110. A sleeve 128 can be disposed over the otologic device 100. For example, the sleeve 128 can be disposed over the angled portion 110 such that a distal portion 130 of the sleeve 128 extends distal of the distal end 118 of the otologic device 100, which can maintain the flexibility of the distal portion 130 of the sleeve 128. In some variants, the sleeve 128 can be advanced proximally along the otologic device 100 until reaching the curve 112. As described herein, the angled portion 110 can direct the positioning of the distal portion 130 of the sleeve 128. A conduit 140 can be disposed along the exterior of the otologic device 100 to the expandable member 132. The conduit 140 can supply gas and/or liquid to inflate the expandable member 132. As illustrated in FIG. 22B, the distal portion 130 can be flexed, which can help avoid damage to the otologic anatomy of a patient during a procedure as the distal portion 130 comes into contact with anatomy of the patient.

The otologic device 100 and/or sleeve 128 can be made of various materials. For example, the otologic device 100 can include metal, metal alloys, polymers, composites, and/or other materials. The sleeve 128 can be made of a flexible material, which can include one or more polymers.

What is claimed is:

1. An otologic device for treatment of an eustachian tube of a patient, comprising:
a detachable sleeve configured for inflation from a first configuration to a second configuration, and
a delivery device comprising a tube, a lumen, a straight portion and an angled portion,
wherein the angled portion extends away from a distal end of the straight portion, the angled portion configured to receive the sleeve thereon that is less rigid than the angled portion, wherein a distal portion of the sleeve is distal of a distal end of the angled portion, the distal portion configured to be disposed in the eustachian tube of the patient.

2. The otologic device of claim 1, wherein the straight portion comprises a tapered section increasing in diameter size in a distal-proximal direction to impede proximal movement of the sleeve past the tapered section.

3. The otologic device of claim 1, wherein the angled portion extends away from the distal end of the straight portion at an angle of 15-45 degrees.

4. The otologic device of claim 1, the delivery device further comprising an open proximal end, an open distal end, and an internal lumen connecting the open proximal end and the open distal end, wherein the open proximal end is configured to receive a medicament that flows through the internal lumen, out the open distal end, and into the distal portion of the sleeve.

5. The otologic device of claim 4, wherein the distal portion of the sleeve comprises perforations configured to deliver the medicament therethrough to the eustachian tube.

6. The otologic device of claim 1, wherein the distal portion of the sleeve comprises an expandable member configured to dilate the eustachian tube.

7. The otologic device of claim 1, wherein the distal portion of the sleeve is configured to deliver a stent to the eustachian tube.

8. The otologic device of claim 1, further configured for use in a sinus cavity.

9. An otologic system for treatment of an eustachian tube of a patient, comprising:
a straight portion; and
an angled portion extending away from a distal end of the straight portion at an angle,
wherein the angle is in a range of 15-50 degrees,
wherein the angled portion has a length in a range of 1-2 centimeters,
the angled portion configured to receive a sleeve thereon that is less rigid than the angled portion, wherein a distal portion of the sleeve is distal of a distal end of the angled portion, the distal portion configured to be disposed in the eustachian tube of a patient.

10. The otologic system of claim 9, further comprising a cylindrical tube shape with an open proximal end, an open distal end, and an internal lumen connecting the open proximal end and the open distal end.

11. The otologic system of claim 9, wherein the distal portion of the sleeve comprises an expandable member configured to dilate the eustachian tube.

12. The otologic system of claim 9, wherein the distal portion of the sleeve is configured to deliver a stent to the eustachian tube.

13. The otologic system of claim 9, wherein the distal portion of the sleeve is flexible to avoid damaging anatomy of the patient.

14. The otologic system of claim 9, further configured for use in a sinus cavity.

15. The otologic system of claim 9, further comprising a navigation sensor configured for placement within the angled portion.

16. A method of treating an eustachian tube of a patient, the method comprising:
disposing a flexible sleeve over a distal end of an otologic device;
proximally advancing the flexible sleeve over an angled portion of the otologic device and a straight portion of the otologic device such that a distal portion of the flexible sleeve is distal of the distal end of the otologic device, the otologic device comprising a tapered section that increases a diameter of the otologic device in a distal-proximal direction to impede further proximal advancement of the flexible sleeve;
inserting the flexible sleeve and the distal end of the otologic device through a nostril of a patient; and
navigating the distal portion of the flexible sleeve to the eustachian tube.

17. The method of claim 16, further comprising expanding a balloon on the distal portion of the flexible sleeve to dilate the eustachian tube.

18. The method of claim 16, further comprising delivering a stent on the distal portion of the flexible sleeve to the eustachian tube.

19. The method of claim 16, further comprising a robotic system configured for delivery of therapy to the eustachian tube.

20. The method of claim 16, wherein the distal portion comprises perforations configured to facilitate passage of medicament from inside the distal portion of the flexible sleeve to the eustachian tube.
